(19) United States
(12) Patent Application Publication
(10) Pub. No.: US 2025/0262143 A1
(43) Pub. Date: Aug. 21, 2025

(54) HARD, DURABLE AND NON-TOXIC UV LIGHT CURABLE SOAK OFF NAIL GEL COMPOSITIONS

(71) Applicant: Allen Johnston, Baltimore, MD (US)

(72) Inventor: Allen Johnston, Baltimore, MD (US)

(21) Appl. No.: 19/057,916

(22) Filed: Feb. 19, 2025

Related U.S. Application Data
(60) Provisional application No. 63/556,264, filed on Feb. 21, 2024.

(51) Int. Cl. A61K 8/81; A61Q 3/02

(52) U.S. Cl. CPC A61K 8/8152; A61Q 3/02; A61K 2800/81

(57) ABSTRACT

A non-toxic UV light-curable methacrylate base coating is applied to a nail, followed by a harder acrylate-based coating. The resulting gel is easily and completely removable from the nail by a short soak in a common solvent such as acetone.

### CROSS-REFERENCE TO RELATED APPLICATION

This is a U.S. non-provisional application claiming the benefit of priority under 35 USC § 119 (e) to the U.S. provisional application U.S. Ser. No. 63/556,264 entitled “Hard, Durable and Non-Toxic UV Light Curable Soak Off Nail Gel Compositions” filed Feb. 21, 2024, the entire disclosure of which is incorporated herein by reference.

### FIELD OF INVENTION

The present invention relates to the ultraviolet (UV) light curable fingernail and toenail coatings. More specifically, the present invention relates to easily removable, UV light curable compositions useful for cosmetically altering nails.

### BACKGROUND

Traditional nail polishes are typically composed of a film-forming polymer such as nitrocellulose dissolved in a volatile organic solvent such as ethyl acetate. The compositions may also include dyes, pigments, plasticizers, adhesion promoters, thickening agents, and UV stabilizers. Nail polish will typically last for a few days before the material starts to chip and will have to be removed and reapplied. The rigidity of the nitrocellulose and the lack of adequate adhesion to the nail plate combine to limit the longevity of the coated nail surface.

A nail gel is a much longer lasting type of nail polish. Nail gels are typically composed of UV light curable acrylate and/or methacrylate monomer(s) or oligomer(s), which, once exposed to ultraviolet light, polymerize into durable (meth)acrylic polymer(s). Unlike a polish, nail gels will typically last for a few weeks if properly applied. The nail gel offers much more protection to the nail, and the compositions may also include dyes and pigments for cosmetic purposes.

As mentioned above, hard, fast curing nail gels compositions are typically composed of (meth)acrylic monomer(s). These compositions have found popularity across Europe due to the easy application and the hard surface coating. However, such compositions are not free of certain drawbacks and deficiencies. One of the negative aspects of these compositions is that many people are allergic to acrylates and contact with acrylates can cause classic allergic contact dermatitis. The dermatitis is usually confined to the exposed area and can cause itching, burning, scaling, hives, and blistering.

In the United States methacrylate-based nail gel compositions are most popular. The major benefit of the methacrylate-based gels is that the methacrylates are less toxicologically hazardous than acrylate systems. Utilization of the right methacrylates in the composition can also create a base coat that can soak off as polymeric sheets in a few minutes in acetone.

However, these compositions also are not free of some deficiencies. For example, methacrylate-based gels are not as fast curing as acrylic based systems, and the surface of the coating is often very tacky. This is caused by the slower reaction kinetics as well as the inhibition of the surface cure due to the presence of oxygen. Methacrylate-based systems require a further preparation step of washing off the air-inhibited layer from the coating with ethanol, ethyl acetate or acetone solvent. Also, as it is well known to reasonably skilled practitioners of the art, removing the gel from the nail plate, when needed is difficult. Typically, the gel breaks into many pieces during the removal process which is very undesirable. It is desired that the gel come off as a single sheet but despite this beneficial property being highly sought, the existing art has been unable to obtain.

It is, therefore, clear, that certain improvements are desired, in order to create nail gels that have all the advantages of the methacrylate-based gels and yet are free of the drawbacks of such gels. The present disclosure describes such improved gels, as well as methods of fabricating and removing them.

### SUMMARY

According to embodiments disclosed herein, methods and compositions are provided for making and easily removing methacrylate- and acrylate-based soak-off nail gels. To accomplish this, compositions comprising a UV light curable nail gel disposed over a nail plate are provided, wherein the gel comprises a methacrylate-based base layer UV light cured onto the nail plate and an acrylate-based topcoat UV light cured onto the methacrylate base coat. The resulting gel can be cleanly and completely removed from the nail plate as a single sheet.

Making the composition begins from preparing a non-toxic base coat composition based on monofunctional and multifunctional methacrylate monomers. It is then followed by the application and curing of a hard topcoat composition based on acrylate monomers. Such products and methods are described with a greater degree of specificity below. Thus, the methacrylate layer would be acting as a protective barrier to prevent the acrylate from contacting the human flesh. While the hard acrylate layer forms a more durable and more aesthetically pleasing surface.

Because of the different properties of the two layers, removal can be achieved much more easily as the acrylate sheet can remain together and the methacrylate layer can be wiped away.

### DETAILED DESCRIPTION

### Terms and Definitions

It is to be understood that both the foregoing general description and the following detailed description are exemplary and explanatory only and are not restrictive of the invention claimed. As used herein, the use of the singular includes the plural unless specifically stated otherwise. As used herein, “or” means “and/or” unless stated otherwise. Furthermore, use of the term “including” as well as other forms, such as “includes,” and “included,” is not limiting. The section headings used herein are for organizational purposes only and are not to be construed as limiting the subject matter described.

Unless specific definitions are provided, the nomenclatures utilized in connection with, and the laboratory procedures and techniques of analytical chemistry, synthetic organic and inorganic chemistry described herein are those known in the art, such as those set forth in “IUPAC Compendium of Chemical Terminology: IUPAC Recommendations (The Gold Book)” (McNaught ed.; International Union of Pure and Applied Chemistry, 2^{nd} Ed., 1997) and “Compendium of Polymer Terminology and Nomenclature: IUPAC Recommendations 2008” (Jones et al., eds; International Union of Pure and Applied Chemistry, 2009). Standard chemical symbols are used interchangeably with the full names represented by such symbols. Thus, for example, the terms “hydrogen” and “H” are understood to have identical meaning. Standard techniques may be used for chemical syntheses, chemical analyses, and formulation.

“About” as used herein means that a number referred to as “about” comprises the recited number plus or minus 1-10% of that recited number. For example, “about” 100 degrees can mean 95-105 degrees or as few as 99-101 degrees depending on the situation. Whenever it appears herein, a numerical range such as “1 to 20” refers to each integer in the given range; e.g., “1 to 20 carbon atoms” means that an alkyl group can contain only 1 carbon atom, 2 carbon atoms, 3 carbon atoms, etc., up to and including 20 carbon atoms (although the term “alkyl” also includes instances where no numerical range of carbon atoms is designated).

“Adhesive” or “adhesive compound” as used herein, refers to any substance that can adhere or bond two items together. Implicit in the definition of an “adhesive composition” or “adhesive formulation” is the fact that the composition or formulation is a combination or mixture of more than one species, component or compound, which can include adhesive monomers, oligomers, and/or polymers along with other materials, whereas an “adhesive compound” refers to a single species, such as an adhesive polymer or oligomer.

More specifically, adhesive composition refers to un-cured mixtures in which the individual components in the mixture retain the chemical and physical characteristics of the original individual components of which the mixture is made. Adhesive compositions are typically malleable and may be liquids, paste, gel or another form that can be applied to an item so that it can be bonded to another item.

“Cured adhesive,” “cured adhesive composition” or “cured adhesive compound” refers to adhesives components and mixtures obtained from reactive curable original compound(s) or mixture(s) thereof which have undergone a chemical and/or physical changes such that the original compound(s) or mixture(s) is (are) transformed into a solid, substantially non-flowing material. A typical curing process may involve crosslinking.

“Curable” means that an original compound(s) or composition material(s) can be transformed into a solid, substantially non-flowing material by means of chemical reaction, crosslinking, radiation crosslinking, or the like. Thus, adhesive compositions of the invention are curable, but unless otherwise specified, the original compound(s) or composition material(s) is (are) not cured.

“Thermoplastic,” as used herein, refers to the ability of a compound, composition or other material (e.g. a plastic) to dissolve in a suitable solvent or to melt to a liquid when heated and freeze to a solid, often brittle and glassy, state when cooled sufficiently.

“Thermoset,” as used herein, refers to the ability of a compound, composition, or other material to irreversibly “cure” resulting in a single three-dimensional network that has greater strength and less solubility compared to the non-cured product. Thermoset materials are typically polymers that may be cured, for example, through heat (e.g., above 200° C.), via a chemical reaction (e.g. epoxy ring-opening, free-radical polymerization, etc or through irradiation (e.g. visible light, UV light, electron beam radiation, ion-beam radiation, or X-ray irradiation).

Thermoset materials, such as thermoset polymers or resins, are typically liquid or malleable forms prior to curing, and therefore may be molded or shaped into their final form, and/or used as adhesives. Curing transforms the thermoset resin into a rigid infusible and insoluble solid or rubber by a cross-linking process. Thus, energy and/or catalysts are typically added that cause the molecular chains to react at chemically active sites (unsaturated or epoxy sites, for example), linking the polymer chains into a rigid, 3-D structure. The cross-linking process forms molecules with a higher molecular weight and resultant higher melting point. During the reaction, when the molecular weight of the polymer has increased to a point such that the melting point is higher than the surrounding ambient temperature, the polymer becomes a solid material.

“Cross-linking,” as used herein, refers to the attachment of two or more oligomer or longer polymer chains by bridges of an element, a molecular group, a compound, or another oligomer or polymer. Crosslinking may take place upon heating or exposure to light; some crosslinking processes may also occur at room temperature or a lower temperature. As cross-linking density is increased, the properties of a material can be changed from thermoplastic to thermosetting.

The term “monomer” refers to a molecule that can undergo polymerization or copolymerization thereby contributing constitutional units to the essential structure of a macromolecule (a polymer).

“Polymer” and “polymer compound” are used interchangeably herein, to refer generally to the combined the products of a single chemical polymerization reaction. Polymers are produced by combining monomer subunits into a covalently bonded chain. Polymers that contain only a single type of monomer are known as “homopolymers,” while polymers containing a mixture of monomers are known as “copolymers.”

As used herein, the term “acrylate” refers to a compound bearing at least one moiety having the structure:

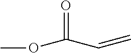

As used herein, the term “methacrylate” refers to a compound bearing at least one moiety having the structure:

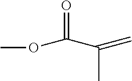

As used herein, the term a “monofunctional methacrylate” is defined by the molecule having one methacrylate unit among other features, and the term a “multifunctional methacrylate” is defined by molecules having more than one methacrylate group among other features.

As used herein, the term “free radical initiator” refers to any chemical species which, upon exposure to sufficient energy (e.g., light, heat, or the like), decomposes into parts, which are uncharged, but every one of such part possesses at least one unpaired electron.

As used herein, the term “coupling agent” refers to chemical species that are capable of bonding to a mineral surface and which also contain polymerizably reactive functional group(s) so as to enable interaction with the adhesive composition.

“Modulus” or “Young's modulus” as used herein, is a measure of the stiffness of a material. Within the limits of elasticity, modulus is the ratio of the linear stress to the linear strain, which can be determined from the slope of a stress-strain curve created during tensile testing.

“Thixotropy” as used herein, refers to the property of a material which enables it to stiffen or thicken in a relatively short time upon standing, but upon agitation or manipulation to change to low-viscosity fluid; the longer the fluid undergoes shear stress, the lower its viscosity. Thixotropic materials are therefore gel-like at rest but fluid when agitated and have high static shear strength and low dynamic shear strength, at the same time.

As used herein, the terms “easily removable” and “completely removable” are used interchangeably to signify the fact that the gel soaked off in acetone can be removed from the nail bed as a single sheet, and that after the single act of removal not more that 5% of the nail bed area can be still covered by the remnants of the original gel; such remnants, if any are still remaining on the nail bed, can be then easily removed by any known method (e.g., scraped off, dissolved, etc.).

### EMBODIMENTS

Embodiments provided below describe two types of formulations to be used in conjunction with each other. There is a methacrylate-based formulation for the base coat, which is applied to the nail first, and an acrylate-based formulation applied after and over the base coat, for the topcoat.

Used in conjunction these two applications form a hard stable nail coating that features two different types of materials, methacrylate(s) forming the base layer or coat and acrylate(s) forming the top layer or coat. Each has different physical properties, modulus, solubility, and they form a boundary within the decorative nail polymer. When the coating is to be removed, the boundary between the two layers provides a point of incursion for the removal solvent, acetone, ethyl acetate and others so that the top layer can be removed in one sheet. Highly crosslinked polymer systems without this boundary must be removed in small pieces after extensive fracturing. The methacrylate base layer is not as well cured as the acrylate top layer due to the air-inhibited layer at the interface of methacrylate polymer and the atmosphere. Atmospheric oxygen terminates the radical chain growth in methacrylate systems leaving the base polymer layer still with a significant content of double bonds.

It is well understood that oxygen interferes with methacrylate polymerization is leaving what is known as an “air inhibited layer” where the polymer is exposed to oxygen. This reaction provides the utility of the process described herein. The base coat is made up of methacrylate containing molecules and is subject to the air inhibition. The cured base coat has the inhibited layer to interface with the topcoat. The topcoat is entirely acrylate-containing molecules which are not subject to the air inhibition. Thus, the acrylate layer acts to reinforce the methacrylate layer on the nail plate. The methacrylate polymer layer has a different solubility in acetone that the acrylate polymer layer. This allows for the removal of the cured nail enhancement through the weaker methacrylate layer as a single unit. This very desired property of easy removability as a single sheet has not been achieved by using any known exiting methods or compositions. The acetone solvent nail enhancement removal solution can penetrate the enhancement through the methacrylate layer.

In view of the foregoing, the preparation of the nail gel begins from forming a UV light curable base coat.

### Base Coat

According to embodiments of the present invention, the base coat is comprised of one or several of methacrylate(s). The methacrylate(s) can be monofunctional or multifunctional or a combination of both. The base coat contains surface active monomers to provide adhesion to the nail plate for the cured coating. Furthermore, the methacrylate molecule(s) being present in the base coat can have portions of lower viscosity and portions of higher viscosity, so that the overall viscosity of the base coat is such that it is appropriate to the nail plate coating technician's requirements.

A variety of methacrylate-based products may be used to form the base coat. One non-limiting example of methacrylate product(s) that can be so used is urethane dimethacrylate product (UDMA) having the following structure:

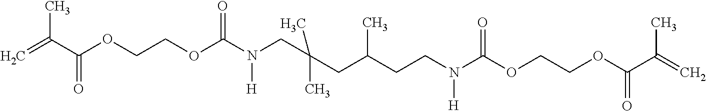 [SMILES: C=C(C)C(=O)OCCOC(=O)NCCC(C)CC(C)(C)CNC(=O)OCCOC(=O)C(=C)C]

If UDMA is so used, it may comprise between about 50 mass % and about 75 mass %, for example, about 66 mass % of the entire base coat.

The base coat has to provide some flexibility when cured and to cure rapidly. To that end, a larger methacrylate containing urethane moieties may be added, if desired, in addition to, or even instead of, UDMA (i.e., to replace a part or all UDMA). One such exemplary urethane-methacrylate product that can be so used is the product designated as “T650 UDMA.” If T650 UDMA is used, it will typically replace only a portion of UDMA so that its contents in the overall base coat be between about 2 mass % and about 12 mass %, for example, about 10 mass %. The product T650 UDMA has the following structure:

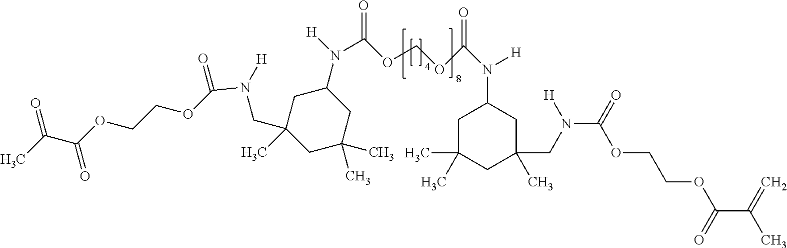

If the base coat is formulated with UDMA and/or T650 modified UDMA, the viscosity may be higher than desired so a dimethacrylate diluent of may be added. One example of a diluent than can be used is a reactive diluent 1,10-decanedimethacrylate (between about 5 mass % and about 20 mass %, for example, about 15 mass % of the entire base coat) having the structure shown below

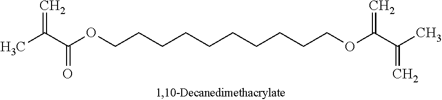 [SMILES: C=C(C)C(=C)OCCCCCCCCCCOC(=O)C(=C)C]

Another exemplary methacrylate-based product that may be used for forming the base coat is pyromellitic dianhydride glycerol dimethacrylate (PMGDM). This product may be useful for improving the adhesion of the base coat to the nail bed. The neat PMGDM, however, is a very viscous product so before being used in the base coat it may beblended with a solution of 2-hydroxpropyl methacrylate (HPMA). The structural formulas of PMGDM and 2-hydroxypropyl methacrylate are as shown below:

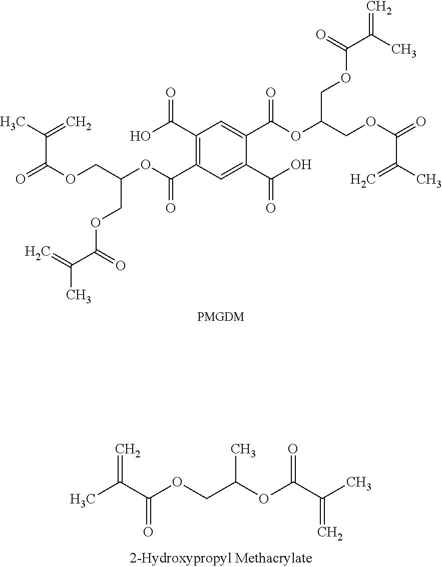

If the blend of PMGDM and 2-hydroxypropyl methacrylate is so used, each of PMGDM and 2-hydroxypropyl methacrylate may comprise between about 2 mass % and about 10 mass %, for example, about 5 mass % of the entire base coat.

Yet another exemplary methacrylate-based product that may be optionally used for making the base coat is the adduct of 2-hydroxyethyl methacrylate phthalic anhydride (HEMA-P) having the following structure

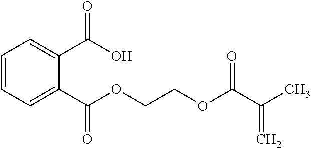 [SMILES: C=C(C)C(=O)OCCOC(=O)c1ccccc1C(=O)O]

If HEMA-P is so used, it may comprise between about 2 mass % and about 10 mass %, for example, about 5 mass % of the entire base coat. The base coat composition also includes a UV light-sensitive photoinitator of free-radical polymerization, such as trimethylbenzoyl diphenylphosphine oxide, (Irgacure TPO) (added under yellow light conditions, at the levels of between about 0.25 mass % and about 3 mass %, for example, less than 1 mass % of the entire base coat), having the structure

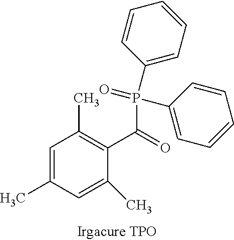 [SMILES: Cc1cc(C)c(C(=O)P(=O)(c2ccccc2)c2ccccc2)c(C)c1]

Furthermore, optionally a small amount of ultramarine blue dye dispersed in UDMA may be used at the 25% (w/w) level with UDMA by a three roll mill. This disperses the dye very finely throughout the monomer.

Since the above-described formulations are free-radically curable materials, they may also optionally contain a small quantity (between about 5,000 ppm (parts per million) and about 50 ppm) of free-radical inhibitors, many of which known as antioxidants. The addition of free-radical inhibitors helps to give the product shelf life and prevent premature curing of the material. One non-limiting example of a free-radical inhibitor that may be optionally used to provide stability over time, is 2,6-di-tert-butyl-4-methylphenol (butylated hydroxytoluene, BHT), with the formula:

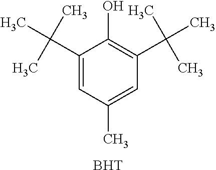

Alternatively, thiols, ascorbic acid, polyphenols, and quinones (such as methoxy hydroquinone (MEHQ) or hydroquinone (HQ)), may be added to formulations, in combination with BHT. The combination of a phenolic inhibitor with benzoquinone derivatives has a synergistic effect and may be a more potent inhibitor mix. The addition of certain nitrosyl compounds, certain tertiary amines, nitro-aromatic compounds are known to those skilled in the art to prevent premature free radical curing.

To control the thixotropy, fine pieces of silica or glass, or both, may be used in the described composition, if desired. Silica and/or glass may be introduced via salinated or unsalinated preparations. For example, a blend of fine particle silica (10% w/w, OX50) and urethane diacrylate (90%) milled together on the three roll mill may be used, to make the formulation able to remain on the prepared nail plate without running. Upon mixing all the material described above, the material can be applied to a substrate nail plate and rapidly cured by exposure to the UV light for between about 5 and about 60 seconds, such as about 20 seconds. Those having ordinary skill in the art will select the appropriate wavelength and the power of the UV radiation source. For example, a 9 W UV lamp with the 265 nm wavelength may be used. The thickness of the base coat can be between about 4 μm and about 6 μm, such as about 5 μm.

### Topcoat

The topcoat is formulated of acrylate products, as opposed to methacrylates used in the base coat as described above. Acrylate monomers tend to react faster with acrylate monomers and methacrylate monomers tend to react faster with methacrylate monomers. The cross reaction is slower. Thus, the application of an acrylate solution on top of a methacrylate layer will lead to a highly cross-linked and extensively reacted polyacrylate layer with a reduced amount of cross reaction. While not desiring to be bound by any particular scientific theory, it may explain a demarcation of reactivity in the nail coating.

Upon acetone exposure, the border of acrylate and methacrylate polymer provides a conduit for acetone insertion. The highly cross-linked poly acrylate layer, therefore, can be dislodged as one sheet, while the more poorly cured methacrylate layer can be washed off with the acetone. This provides a valuable attribute for nail decorative coatings because the coating is dislodged in one sheet from the nail plate. Pure acrylate or pure methacrylate systems are dislodged in small pieces as they fracture upon removal.

The remaining base coat can be solubilized in the acetone and removed with an acetone wipe if any remains after the topcoat is dislodged.

The topcoat is formulated with acrylate-based materials in a fashion that is similar to that used to formulate the base coat, but instead of methacrylate products used to make the base coat, corresponding acrylates can be used.

Accordingly, urethane diacrylate product (UDA) may be used (between about 50 mass % and about 75 mass %, for example, about 66 mass % of the entire topcoat), UDA having the following structure:

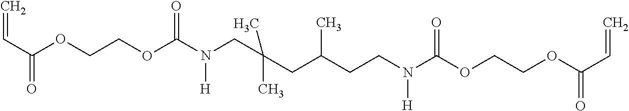 [SMILES: C=CC(=O)OCCOC(=O)NCCC(C)CC(C)(C)CNC(=O)OCCOC(=O)C=C]

Because a hard finish is desired, it is also advisable to use the T650 urethane diacrylate (“T650 UDA”) to replace all or a portion of UDA so that the contents of T650 UDA in the overall base coat be between about 2 mass % and about 12 mass %, for example, about 10 mass %. The product T650 UDA has the following structure:

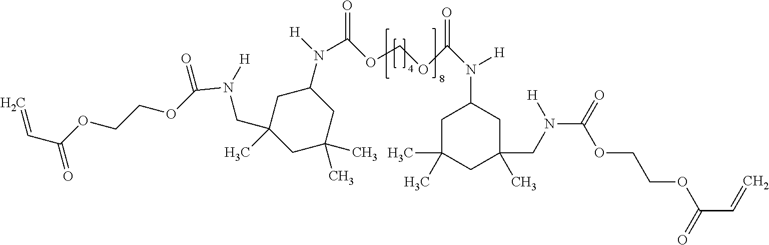

The viscosity of the base coat may be controlled by using the C-10 diacrylate dilutant, in the same fashion and quantities as described for 1,10-decanedimethacrylate used for the same purpose in the base coat.

Other acrylic products that can be used for making the topcoat may be the adduct of isophorone diisocyanate and 2-hydroxyethyl acrylate (IDPI-HEA) and UDA with 25% (w/w) ultramarine blue dye milled as the methacrylate dye solution. The IPDI-HEA contributes to raises the viscosity of the formulation.

The same initiator (i.e., Irgacure TPO solid) described above for the base formulation may be used, in the same quantities for making the topcoat formulation. It is worth mentioning that many of the free-radical generating photoinitators work synergistically in the presence of a tertiary amine compound to act as a photoreductant. It has been found that the tertiary amines that are most affective in radical polymerization are the ones that contain at least one aromatic group. Primary and secondary amines often act as inhibitors in the free-radical reaction. Ethyl-4-dimethylaminobenzoate (structure shown below) was found to be a tertiary amine that was very affective in the UV light cured formulation.

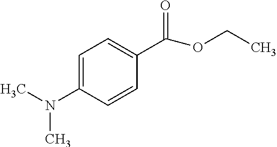

The formulations that are the subject of the present disclosure, however, are not limited to only this compound; many other tertiary aromatic amine compounds are also contemplated for use in the practice of the invention. The amount of tertiary aromatic amine can range from 0.1 to about 15% by weight of the formulation.

It is known to those skilled in the art that all the acrylated and methacrylated urethane derivatives described above may be readily prepared by the reaction of the corresponding isocyanate species with a hydroxyalkylacrylate or hydroxyalkylmethacrylate in the presence of an appropriate catalyst such as dibutyltin dilaurate. Many other metal carboxylates may also be used to produce urethanes such as aluminum, bismuth, zinc, and zirconium. Tertiary amines such as triethylenediamine, 1,4-diazabicyclo[2.2.2]octane, dimethylcyclohexylamine, and dimethylethanolamine are also used to prepare the acrylated urethanes. In some circumstances no catalyst is required.

Hydroxyethyl methacrylate (HEMA) and hydroxyethyl acrylate (HEA) are the most readily available and lowest cost material for use in price sensitive formulations. However, other material such as hydroxypropyl-, hydroxybutyl-, hydroxypentyl-, and longer chain hydroxyalkyl (meth)acrylates are also contemplated for use in the practice of the invention.

Isophorone diisocyanate is the starting material used in the preparation of the invention compounds. This material has two distinct isocyanate groups, an isocyanate attached to a primary carbon and one attached to a secondary carbon. It has been determined that under the reaction conditions with dibutyltin dilaurate as the catalyst the secondary isocyanate reacts at a much higher rate than the primary isocyanate.

The reaction of two moles of isophorone diisocyanate with one mole of poly-THF using dibutyltin dilaurate as the catalyst produces the following intermediate.

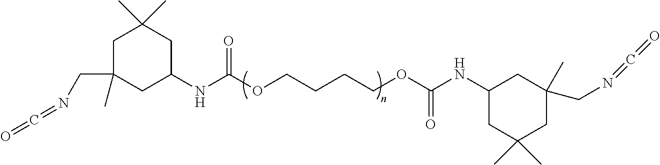

The subsequent addition of two moles of hydroxyethyl methacrylate or hydroxyethyl acrylate will react with the remaining isocyanate groups and produce the desired product.

Polytetrahydrofuran, also called poly(tetramethylene ether) glycol or poly(tetramethylene oxide), is a chemical compound with formula (C₄H₈O)ₙOH₂ or HO—(—(CH₂)₄O—)ₙ—H. It can be viewed as a polymer of tetrahydrofuran, or as the polyether derived from 1,4-butanediol. The product is commercially available as polymers of low average molecular weights, between 250 and 3000 Daltons. In this form it is a white waxy solid that melts between 2° and 30° C. The product is sold under various trade names including Terathane® and PolyTHF®. All the molecular weight variations are contemplated for use in the practice of the invention. Useful Tetrathane® oligomers are available from Invista (Wichita, Kansas) and are illustrated below.

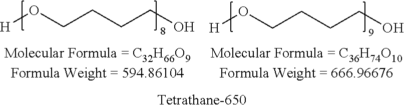

As can be seen Tetrathane®-650 products are used in preparing the above-described T650 modified UDMA and UDA products.

Depending upon the formulation and whether it is a topcoat or base coat, the amine concentration may vary from 0 through 5%. Some formulations did not perform well with the amine present and turned undesirable colors. Those formulations succeeded with further modifications to the monomer content and removal of the amine.

An additional ingredient added to the formulation includes compound such as hydroxyethyl (meth)acrylate. Not to be bound by any theory, but these compounds are often added to boost the adhesion of the material to various substrates or to modify the viscosity of the formulation.

Also contemplated in the practice of the invention is the addition of colorants, such as dyes and pigments. The addition of these types of compounds to the topcoat or base coat formulations would create a more aesthetically pleasing coating for the benefit of the animal and the owner.

Iron oxides: These inorganic pigments are used in all types of cosmetics and have three basic shades: black, yellow and red. By blending these three oxides in the right proportions you can produce an array of browns, tans, and umbers. By careful blending, an array of natural looking flesh tones may be produced.

Chromium dioxides: The Chromium dioxides are used for most categories of cosmetic preparations. Colors range from dull olive green to a blue green, or bright green.

Ultramarines: Ultramarines vary in shade from bright blue to violet, pink and even green. These pigments are also based on lazurite various shades and are contemplated for use in the practice of the invention.

Manganese violet: This brightly colored inorganic pigment is purple colored, based on ammonium manganese diphosphate.

Iron blue: This very deep intense dark blue pigment is widely used in all cosmetic applications. Ferric ammonium ferrocyanide and lazurite are contemplated for use in making blue colored product.

White pigments: White pigments are widely used in all cosmetics; they have extremely good covering power and are almost totally inert in addition to being extremely stable to heat and light. Titanium dioxide and zinc oxide are the most used in cosmetics. Other known white pigments contemplated for use in the practice of the invention include but are not limited to compounds such as: aluminum; aluminum hydroxide sulphate; aluminum silicate; barium sulphate; bismuth chloride oxide; calcium carbonate; calcium sulphate; magnesium carbonate; aluminum stearate; magnesium stearate; zinc stearate; and calcium stearate.

Mica: Chemically mica is potassium aluminum silicate dihydrate. It is mined as the ore muscovite and occurs in multi-layered bright translucent sheets.

Black: The color black is typically derived from carbon black and is also contemplated for use in the practice of the invention.

In addition to inorganic pigments there are numerous organic dyes that are also contemplated for use in the practice of the invention. These materials are available in various colors and are known to those skilled in the art.

Color stabilizers such as dihydroxyethylbenzophenone are also contemplated for use in the practice of the invention.

Upon mixing all the material described above, the material can be applied to the previously cured base coat followed by rapidly curing of the topcoat by exposure to the UV light for between about 5 and about 60 seconds, such as about 20 seconds. Again, 9 W UV lamp with the 265 nm wavelength may be used, but those having ordinary skill in the art may select a different wavelength and the power of the UV radiation source. The thickness of the topcoat can be between about 4 μm and about 6 μm, such as about 5 μm.

As mentioned above, to remove the nail gel, it can be exposed to acetone. Upon acetone exposure, and after the gel gets soaked in acetone, the cured formulation can be easily removed from the nail plate by removal of the gel as a single sheet of material.

### EXAMPLES

To illustrate some features and advantages of the embodiments disclosed herein, the following examples of formulations actually prepared are provided. Compositions were made by mixing components in the quantities indicated below as mass percentages.

### Example 1

Base Coat Formulation

| ITEM | amount |
|---|---|
| BHT | 298 ppm |
| E4DMAB | 1.34% |
| Blue tint T650 UDMA | 2.38% |
| HEMA-P | 2.35% |
| T650 UDMA | 29.93% |
| UDMA | 60.45% |
| HEMA Acetate | 2.23% |
| TPO (solid) | 1.28% |

### Example 2

Pink Base Coat Formulation

| ITEM | amount |
|---|---|
| BHT | 700 ppm |
| Pink Dye in UDMA | 0.067% |
| HEMA-P | 2.00% |
| T650 UDMA | 66.27% |
| UDMA | 10.00% |
| HEMA Acetate | 20.00% |
| TPO (solid) | 1.00% |

### Example 3

Topcoat

| ITEM | amount |
|---|---|
| BHT | 668 ppm |
| E4DMAB | 3.00% |
| Blue tint T650 UDA | 5.34% |
| T650 UDA | 10.08% |
| UDA | 73.88% |
| HEA Acetate | 5.01% |
| TPO (solid) | 2.67% |

### Example 4

Pink Topcoat

| ITEM | amount |
|---|---|
| BHT | 200 ppm |
| Pink Dye in UDA | 1.33% |
| T650 UDA | 20.00% |
| UDA | 74.60% |
| HEA Acetate | 0.67% |
| TPO (solid) | 3.33% |

### Example 5

Tip White Coat

| ITEM | amount |
|---|---|
| BHT | 200 ppm |
| Blue tint UDA | 2.50% |
| TiO₂ UDA | 21.47% |
| UDA | 73.33% |
| TPO (solid) | 2.50% |

### Example 6

Clear, Colorless Basecoat

| ITEM | Amount |
|---|---|
| BHT | 700 ppm |
| Blue dye in UDMA | 2.07% |
| HEMA-P | 2.00% |
| T650 UDMA | 64.87% |
| UDMA | 10.00% |
| HEMA Acetate | 20.00% |
| TPO solid | 1.00% |

### Example 7

Clear, Colorless Topcoat

| ITEM | Amount |
|---|---|
| Blue dye in UDA | 0.07% |
| TPO solid | 0.93% |
| T650 UDA | 52.00% |
| 10% OX 50 silica in UDA | 12.00% |
| HEA-IPDI | 1.00% |
| UDA | 10.00% |
| C10 diacrylate | 24.00% |

### Example 8

Clear, Colorless Topcoat

| ITEM | Amount |
|---|---|
| Blue dye in UDA | 0.07% |
| TPO solid | 0.93% |
| T650 UDA | 55.00% |
| 10% OX 50 silica in UDA | 10.00% |
| HEA-IPDI | 2.53% |
| UDA | 7.53% |
| C10 diacrylate | 23.93% |

### Example 9

Clear, Colorless Basecoat

| Item | Loading |
|---|---|
| UDMA | 66.69% |
| C-10 Dimethacrylate | 15.67% |
| T650 Urethane Dimethacrylate | 8.67% |
| 50% PMGDM/50% HPMA blend | 8.00% |
| TPO (solid) | 0.93% |
| Methacrylate Blue dye | 0.03% |

### Example 10

Clear, Colorless Topcoat

| Item | Loading |
|---|---|
| T650 Urethane Diacrylate | 56.69% |
| C-10 Diacrylate | 23.94% |
| 10% Aerosil Acrylate blend | 10.00% |
| Urethane Diacrylate | 5.87% |
| IDPI-HEA Adduct | 2.53% |
| TPO (solid) | 0.93% |
| Acrylate Blue Dye | 0.07% |

The pigmentation of the formulation was introduced via a suspension of the pigment in monomer. The pigment was milled into the monomer with a three roll mill. Creating a dispersion of pigment in monomer through this method allowed for an easier and more uniform dispersion of the pigment in the final gel. The pigment particles were very small and resisted dispersion from agglomerates that form in the isolated pigment. The three-roll mill provided sufficient shear force to break up the agglomerates and allow them to be completely wetted by the monomer. Once wetted with the monomer, the agglomerates cannot reform, and the pigment was then evenly distributed throughout the formulation.

While the embodiments of this invention have been described with respect to these specific examples for purposes of clarity and illustration, it should be clear that those having ordinary skill in the art, in light of the present disclosure, may be able to devise modifications and variations without departing from the spirit or scope of the appended claims.

What is claimed is:

1. A composition, comprising a UV light curable nail gel disposed over a nail plate, the gel comprising:
(a) a methacrylate-based base layer UV light cured onto the nail plate, and
(b) an acrylate-based topcoat UV light cured onto the methacrylate base coat,
with the further proviso that the gel can be removed from the nail plate as a single sheet.

2. The composition of claim 1, wherein the methacrylate is selected from the group consisting of urethane dimethacrylate (UDMA), 2-hydroxyethyl methacrylate phthalic anhydride (HEMA-P), pyromellitic dianhydride glycerol dimethacrylate (PMGDM), 2-hydroxypropyl methacrylate, T650 urethane dimethacrylate (T650 UDMA), 1,10-decanedimethacrylate, and any combination thereof.

3. The composition of claim 2, further comprising a quantity of a free-radical inhibitor.

4. The composition of claim 3, wherein the free-radical inhibitor is butylated hydroxytoluene (BHT).

5. The composition of claim 2, further comprising a quantity of an initiator of free-radical polymerization.

6. The composition of claim 5, wherein the initiator is trimethylbenzoyl diphenylphosphine oxide (TPO).

7. The composition of claim 1, wherein the acrylate is selected from the group consisting of urethane diacrylate (UDA), T650 urethane diacrylate (T650 UDA), 1,10-decanediacrylate, and any combination thereof.

8. The composition of claim 7, further comprising a quantity of a free-radical inhibitor.

9. The composition of claim 8, wherein the free-radical inhibitor is butylated hydroxytoluene (BHT).

10. The composition of claim 7, further comprising a quantity of an initiator of free-radical polymerization.

11. The composition of claim 10, wherein the initiator is trimethylbenzoyl diphenylphosphine oxide (TPO).

12. The composition of claim 1, further comprising a quantity of a pigment or a dye for imparting a desired color to the composition.

13. The composition of claim 12, wherein the pigment or the dye is independently selected from the group consisting of iron oxides, chromium dioxides, ultramarines, manganese violet, iron blue, titanium dioxide, zinc oxide, aluminum, aluminum hydroxide sulphate, aluminum silicate, barium sulphate, bismuth chloride oxide, calcium carbonate, calcium sulphate, magnesium carbonate, aluminum stearate, magnesium stearate, zinc stearate, calcium stearate, mica, carbon black, organic dyes, and any combination thereof.

14. A method for removing the gel of claim 1 from the nail bed, comprising soaking the gel in acetone and removing the gel as a single sheet thereby.
